(19) United States
(12) Patent Application Publication
(10) Pub. No.: US 2006/0264939 A1
(43) Pub. Date: Nov. 23, 2006

(54) Interspinous process implant with slide-in distraction piece and method of implantation

(75) Inventors: James F. Zucherman, San Francisco, CA (US); Ken Y. Hsu, San Francisco, CA (US); Henry Klyce, Piedmont, CA (US); Charles J. Winslow, Walnut Creek, CA (US); Scott A. Yerby, Montara, CA (US); John J. Flynn, West Milford, NJ (US); Steven T. Mitchell, Pleasant Hill, CA (US); John A. Markwart, Castro Valley, CA (US)

Correspondence Address: FLIESLER MEYER, LLP, FOUR EMBARCADERO CENTER, SUITE 400, SAN FRANCISCO, CA, 94111, US

(73) Assignee: St. Francis Medical Technologies, Inc., Alameda, CA (US)

(21) Appl. No.: 11/384,055

(22) Filed: Mar. 17, 2006

Related U.S. Application Data
(63) Continuation-in-part of application No. 10/850,267, filed on May 20, 2004.
(60) Provisional application No. 60/664,049, filed on Mar. 22, 2005.
(60) Provisional application No. 60/472,817, filed on May 22, 2003.

(51) Int. Cl. A61F 2/30

(52) U.S. Cl. 606/61

(57) ABSTRACT

Systems and method in accordance with embodiments of the present invention can includes an implant having an initiating piece and a distraction piece. The initiating piece can include a lower distraction element, a lower portion of a second wing, a lower portion of a spacer, and a lower portion of a first wing. The initiating piece can be positioned such that an interspinous ligament of the targeted motion segment is disposed between the first and second wing. The distraction piece can include an upper distraction element, an upper portion of a second wing, an upper portion of the spacer, and an upper portion of the first wing, and can be mated with the initiating piece by mating a rail of the distraction piece with a slot of the initiating piece, or the implant is disposed between adjacent spinous processes.

### CLAIM OF PRIORITY

This application claims priority to U.S. Provisional Application No. 60/664,049 entitled INTERSPINOUS PROCESS IMPLANT WITH SLIDE-IN DISTRACTION PIECE AND METHOD OF IMPLANTATION, by Zucherman et al, filed Mar. 22, 2005, (Attorney Docket No. KLYC-1087US4) and is a continuation-in-part of U.S. patent application Ser. No. 10/850,267 entitled DISTRACTIBLE INTERSPINOUS PROCESS IMPLANT AND METHOD OF IMPLANTATION, by Zucherman et al, filed May 20, 2004, (Attorney Docket No. KLYC01087US2) which claims priority to U.S. Provisional Patent Application No. 60/472,817 entitled CERVICAL INTERSPINOUS PROCESS DISTRACTION IMPLANT AND METHOD OF IMPLANTATION, by Zucherman et al., filed May 22, 2003, (Attorney Docket No. KLYC-01087US0).

### CROSS-REFERENCE TO RELATED APPLICATIONS

This U.S. Patent Application incorporates by reference all of the following co-pending applications and issued patents:

U.S. Patent Application Ser. No. 60/664,049, entitled “Interspinous Process Implant With Slide-In Distraction Piece and Method of Implantation,” (Attorney Docket Number KLYC-01087US5) filed concurrently;

U.S. Pat. No. 6,419,676, entitled “Spine Distraction Implant and Method,” issued Jul. 16, 2002 to Zucherman, et al.;

U.S. Pat. No. 6,451,019, entitled “Supplemental Spine Fixation Device and Method,” issued Sep. 17, 2002 to Zucherman, et al.;

U.S. Pat. No. 6,582,433, entitled “Spine Fixation Device and Method,” issued Jun. 24, 2003 to Yun;

U.S. Pat. No. 6,652,527, entitled “Supplemental Spine Fixation Device and Method,” issued Nov. 25, 2003 to Zucherman, et al;

U.S. Pat. No. 6,695,842, entitled “Interspinous Process Distraction System and Method with Positionable Wing and Method,” issued Feb. 24, 2004 to Zucherman, et al;

U.S. Pat. No. 6,699,246, entitled “Spine Distraction Implant,” issued Mar. 2, 2004 to Zucherman, et al; and

U.S. Pat. No. 6,712,819, entitled “Mating Insertion Instruments for Spinal Implants and Methods of Use,” issued Mar. 30, 2004 to Zucherman, et al.

### TECHNICAL FIELD

This invention relates to interspinous process implants.

### BACKGROUND OF THE INVENTION

The spinal column is a bio-mechanical structure composed primarily of ligaments, muscles, vertebrae and intervertebral disks. The bio-mechanical functions of the spine include: (1) support of the body, which involves the transfer of the weight and the bending movements of the head, trunk and arms to the pelvis and legs, (2) complex physiological motion between these parts, and (3) protection of the spinal cord and the nerve roots.

As the present society ages, it is anticipated that there will be an increase in adverse spinal conditions which are characteristic of older people. By way of example only, with aging comes an increase in spinal stenosis (including, but not limited to, central canal and lateral stenosis), and facet arthropathy. Spinal stenosis results in a reduction foraminal area (i.e., the available space for the passage of nerves and blood vessels) which compresses the cervical nerve roots and causes radicular pain. Humpreys, S. C. et al., Flexion and traction effect on C5-C6 foraminal space, Arch. Phys. Med. Rehabil., vol. 79 at 1105 (September 1998). Another symptom of spinal stenosis is myelopathy, which results in neck pain and muscle weakness. Id. Extension and ipsilateral rotation of the neck further reduces the foraminal area and contributes to pain, nerve root compression and neural injury. Id.; Yoo, J. U. et al., Effect of cervical spine motion on the neuroforaminal dimensions of human cervical spine, Spine, vol. 17 at 1131 (Nov. 10, 1992). In contrast, neck flexion increases the foraminal area. Humpreys, S. C. et al., at 1105.

Pain associated with stenosis can be relieved by medication and/or surgery. It is desirable to eliminate the need for major surgery for all individuals, and in particular, for the elderly.

Accordingly, a need exists to develop spine implants that alleviate pain caused by spinal stenosis and other such conditions caused by damage to, or degeneration of, the cervical spine. Such implants would distract, or increase the space between, the vertebrae to increase the foraminal area and reduce pressure on the nerves and blood vessels of the cervical spine.

A further need exists for development of a minimally invasive surgical implantation method for cervical spine implants that preserves the physiology of the spine.

Further, a need exists for an implant that accommodates the distinct anatomical structures of the spine, minimizes further trauma to the spine, and obviates the need for invasive methods of surgical implantation. Additionally, a need exists to address adverse spinal conditions that are exacerbated by spinal extension.

### BRIEF DESCRIPTION OF THE DRAWINGS

Further details of embodiments of the present invention are explained with the help of the attached drawings in which:

FIG. 1 is a perspective view of an embodiment of an implant in accordance with the present invention having a spacer, a distraction guide, and a wing with an elliptical cross-section.

FIG. 2 is an end view of the implant of FIG. 1.

FIG. 3 is a perspective view of another embodiment of an implant in accordance with the present invention having a wing with a teardrop-shaped cross-section.

FIG. 4 is an end view of a second wing for use with the implant of FIG. 3.

FIG. 5 is a perspective view of an embodiment of an implant in accordance with the present invention having a rotatable spacer and a wing with an elliptical cross-section.

FIG. 6 is a perspective view of an embodiment of an implant in accordance with the present invention having a rotatable spacer with two wings that are teardrop-shaped in cross-section.

FIG. 7 depicts the axis of rotation of the implant of FIG. 6 as seen from an end view.

FIG. 8 is a perspective view of an embodiment of an implant in accordance with the present invention having a wing that is truncated at a posterior end.

FIG. 9A is an end view of the implant of FIG. 8.

FIG. 9B is a truncated second wing for use with the implant of FIG. 9A.

FIG. 10 is a plan view of an embodiment of an implant in accordance with the present invention wherein a screw is used to secure a second wing to the spacer.

FIG. 11 is a perspective view of the second wing of FIG. 10.

FIG. 12 is a perspective view of the implant of FIG. 10.

FIG. 13A is a front view of a second wing for use with some embodiments of implants of the present invention having a flexible hinge mechanism for securing the second wing to an implant.

FIG. 13B is a side-sectional view of the second wing of FIG. 13A.

FIG. 14A is a plan view of an embodiment of an implant for use with the second wing of FIGS. 13A and 13B.

FIG. 14B is a front view of the second wing of FIGS. 13A and 13B.

FIG. 15A is a top view of an embodiment of an implant in accordance with the present invention positioned between spinous processes of adjacent cervical vertebrae.

FIG. 15B is a top view of the implant of FIG. 15A showing wing orientation.

FIG. 16 is a top view of two such implants of the invention of FIGS. 15A and 15B, positioned in the cervical spine.

FIG. 17 is a side view of two implants of the invention positioned in the cervical spine, with stops or keeps at the proximal ends of the spinous processes.

FIG. 18 is a perspective view of an alternative embodiment of an implant for use with systems and methods of the present invention, the implant including an distraction piece mated with a initiating piece.

FIG. 19A is a perspective view of the initiating piece of the implant of FIG. 18.

FIG. 19B is a perspective view of a proximal end of an insertion tool having prongs positioned within cavities of the initiating piece.

FIG. 19C is a perspective view of the prongs arranged in a locked position within the cavities of the initiating piece.

FIGS. 20A-20D are posterior views of the initiating piece of FIG. 19A as the initiating piece is urged into position with the interspinous ligament disposed between the first wing and the second wing.

FIG. 21 is a perspective view of the slide-in distraction piece of the implant of FIG. 18.

FIGS. 22A-22D are posterior views showing the slide-in distraction piece of FIG. 21 mating with the initiating piece positioned as shown in FIG. 20D so that an implant as shown in FIG. 18 is disposed between the adjacent spinous processes.

FIG. 23A illustrates an embodiment of a method in accordance with the present invention for implanting the interspinous implant of FIGS. 1-17.

FIG. 23B illustrates an embodiment of a method in accordance with the present invention for implanting the interspinous implant of FIG. 18.

### DETAILED DESCRIPTION INTERSPINOUS IMPLANTS

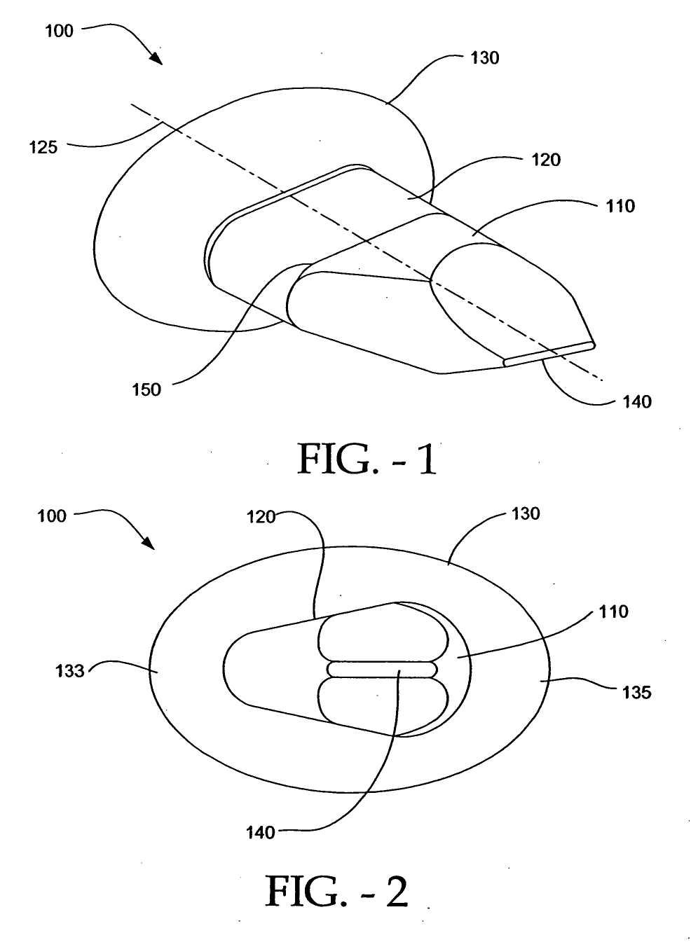

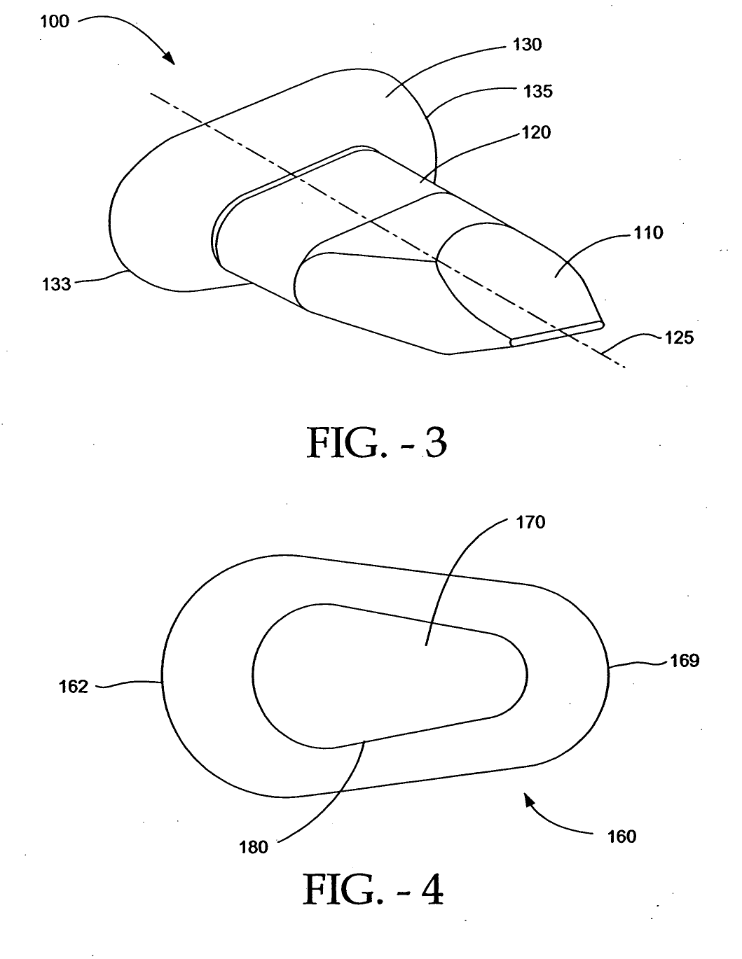

FIGS. 1 and 2 illustrate an implant 100 in accordance with an embodiment of the present invention. The implant 100 comprises a wing 130, a spacer 120, and a lead-in tissue expander (also referred to herein as a distraction guide) 110. The distraction guide 110 in this particular embodiment is wedge-shaped, i.e., the implant has an expanding cross-section from a distal end of the implant 102 to a region 104 where the guide 110 joins with the spacer 120 (referencing for the figures is based on the point of insertion of the implant between spinous processes). As such, the distraction guide functions to initiate distraction of the soft tissue and the spinous processes when the implant 100 is surgically inserted between the spinous processes. It is to be understood that the distraction guide 110 can be pointed and the like, in order to facilitate insertion of the implant 100 between the spinous processes of adjacent cervical vertebrae. It is advantageous that the insertion technique disturb as little of the bone and surrounding tissue or ligaments as possible in order to reduce trauma to the site and promote early healing, and prevent destabilization of the normal anatomy. In the embodiment of FIGS. 1 and 2, there is no requirement to remove any of the bone of the spinous processes and no requirement to sever or remove from the body ligaments and tissues immediately associated with the spinous processes. For example, it is unnecessary to sever the ligamentum nuchae (supraspinous ligament), which partially cushions the spinous processes of the upper cervical vertebrae.

As can be seen in FIGS. 1-3, the spacer 120 can be teardrop-shaped in cross-section perpendicular to a longitudinal axis 125 of the implant 100. In this way, the shape of the spacer 120 can roughly conform to a wedge-shaped space, or a portion of the space, between adjacent spinous processes within which the implant 100 is to be positioned. In other embodiments, the spacer 120, can have alternative shapes such as circular, wedge, elliptical, ovoid, football-shaped, and rectangular-shaped with rounded corners and other shapes, and be within the spirit and scope of the invention. The shape of the spacer 120 can be selected for a particular patient so that the physician can position the implant 100 as close as possible to the anterior portion of the surface of the spinous process. The shape selected for the spacer 120 can affect the contact surface area of the implant 100 and the spinous processes that are to be subject to distraction. Increasing the contact surface area between the implant 100 and the spinous processes can distribute the force and load between the spinous frame and the implant 100.

As can be seen in FIGS. 1 and 2, the wing 130 in an embodiment can be elliptically shaped in cross-section perpendicular to the longitudinal axis 125. The dimensions of the wing 130 can be larger than that of the spacer 120, particularly along the axis of the spine, and can limit or block lateral displacement of the implant 100 in the direction of insertion along the longitudinal axis 125. As illustrated in the embodiment of FIG. 3, the wing 130 can alternatively have other cross-sectional shapes, such as teardrop, wedge, circular, ovoid, football-shaped, and rectangular-shaped with rounded corners and other shapes, and be within the spirit and scope of the invention. The wing 130 has an anterior portion 138 and a posterior portion 136.

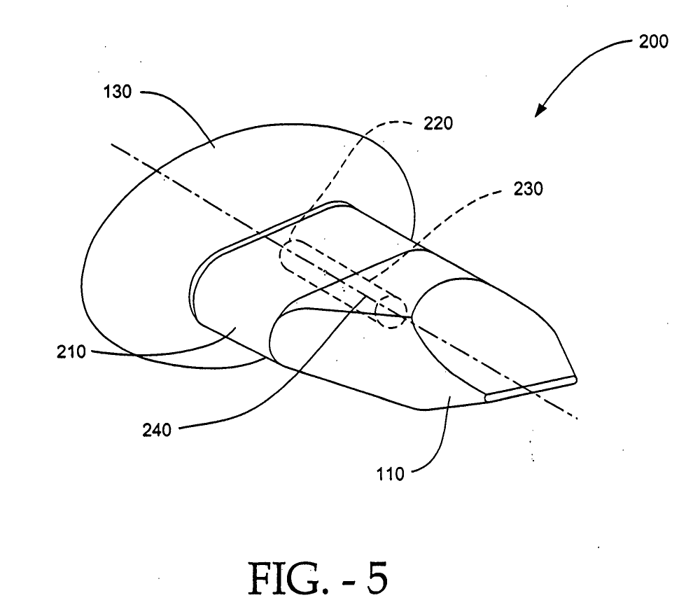

In other embodiments, the implant 100 can include two wings, with a second wing 160 (shown in FIG. 4) separate from the distraction guide 110, spacer 120 and first wing 130. The second wing 160 can be connected to the distal end of the spacer 120. The second wing 160, similar to the first wing 130, can limit or block lateral displacement of the implant 100, however displacement is limited or blocked in the direction along the longitudinal axis 125 opposite insertion. When both the first wing 130 and the second wing 160 are connected with the implant 100 and the implant 100 is positioned between adjacent spinous processes, a portion of the spinous processes can be sandwiched between the first wing 130 and the second wing 160, limiting any displacement along the longitudinal axis 125.

As can be seen in FIG. 4, the second wing 160 can be teardrop-shaped in cross-section. The wider end 166 of the second wing 160 is the posterior end and the narrower end 168 of the second wing 160 is the anterior end. Unlike the first wing 130, however, an opening 164 is defined within the second wing 160, the opening 164 being at least partially circumscribed by a lip 162 that allows the second wing 160 to pass over the distraction guide 110 to meet and connect with the spacer 120. The second wing 160 can be secured to the spacer 120 once the second wing 160 is properly positioned. The second wing 160 can be connected with the implant after the implant 100 is positioned between the spinous processes.

It is to be understood that the implant can be made in two pieces. The first piece can include the first wing 130, the spacer 120, and the distraction guide 110. The second piece can include the second wing 160. Each piece can be manufactured using technique known in the art (e.g., machining, molding, extrusion). Each piece, as will be more fully discussed below, can be made of a material that is bio-compatible with the body of the patient. An implant can be formed with multiple pieces and with the pieces appropriately joined together, or alternatively, an implant can be formed as one piece or joined together as one piece.

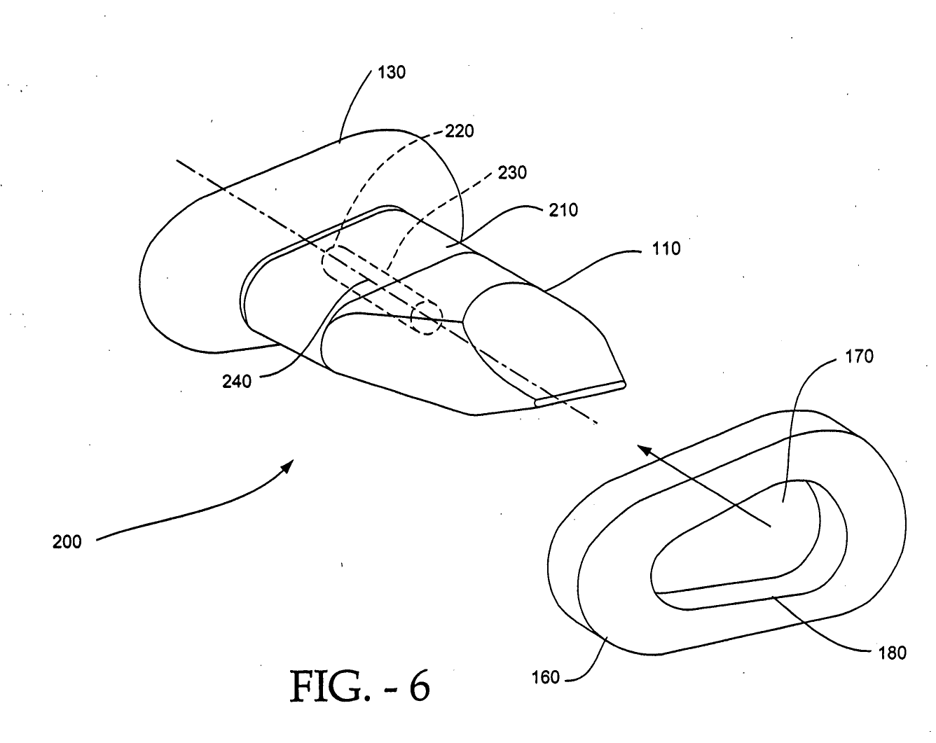

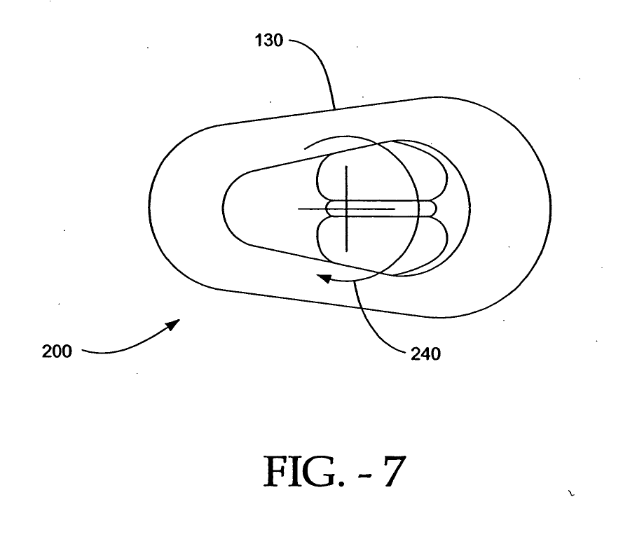

Further embodiments of implants in accordance with the present invention are depicted in FIGS. 5-7. In such embodiments, the spacer 220 can be rotatable about the longitudinal axis 225 relative to the first wing 130, or relative to the first wing 130 and a second wing 160 where two wings are used. The spacer 220 can be rotatable or fixed relative to the distraction guide 110. Where the spacer 220 is rotatable relative to the distraction guide 110, the spacer 220 can include a bore 222 running the length of the longitudinal axis 225, and a shaft 224 inserted through the bore 222 and connecting the distraction guide 110 with the first wing 130. It can be advantageous to position any of the implants taught herein as close as possible to the vertebral bodies. The rotatable spacer 220 can rotate to conform to or settle between adjacent spinous processes as the implant 200 is inserted and positioned during implantation, so that on average the contact surface area between the spacer 220 and the spinous processes can be increased over the contact surface area between a fixed spacer 120 and the spinous processes. Thus, the rotatable spacer 220 can improve the positioning of the spacer 220 independent of the wings 130,160 relative to the spinous processes. The embodiment of FIG. 6 includes a teardrop-shaped first wing 130, and a teardrop-shaped second wing 160, similar to the second wing 160 depicted in the embodiment of FIG. 3. As discussed below, the shape of the wings 130,160 in FIGS. 3 and 6 is such that the implants 100,200 accommodate the twisting of the cervical spine along its axis, for example, as the head of a patient turns from side to side.

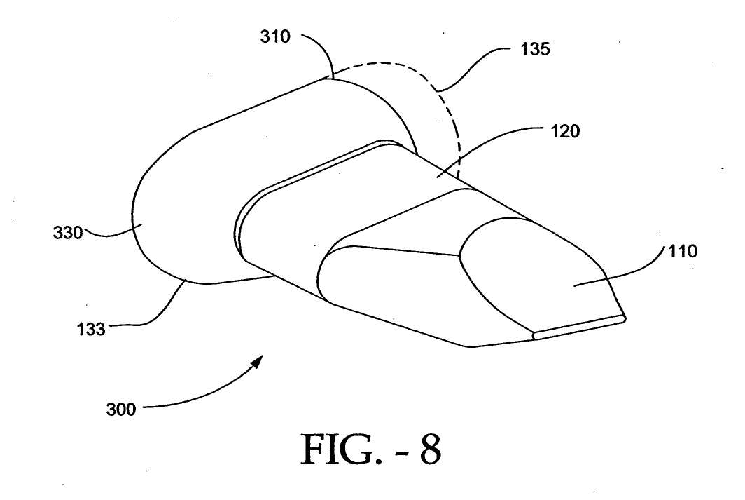

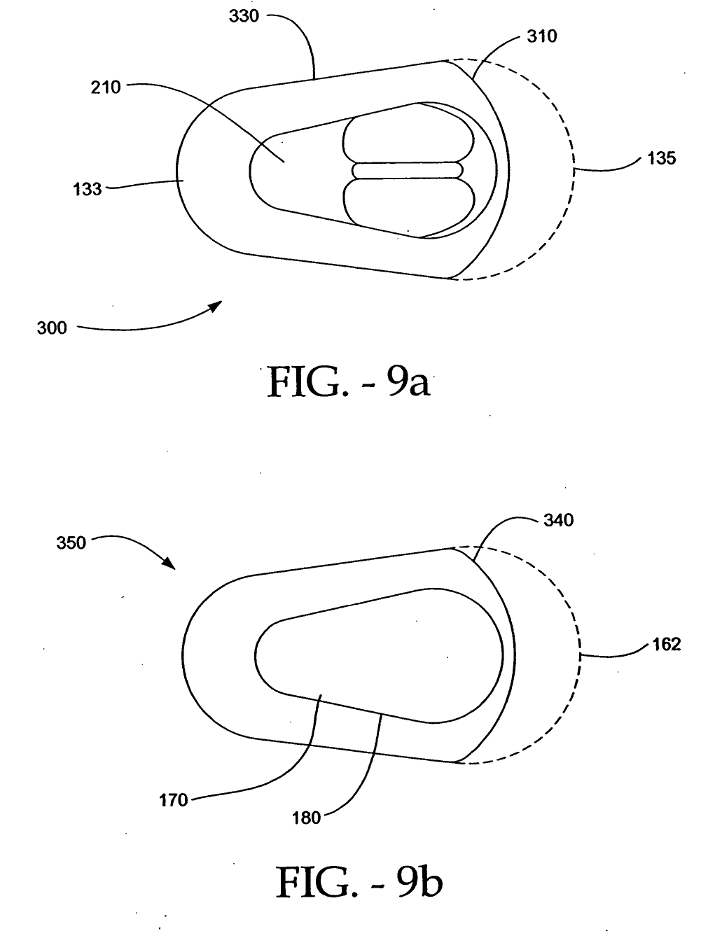

FIG. 8 is a perspective view and FIG. 9A is an end view of still another embodiment of an implant in accordance with the present invention, wherein the posterior portion 336 of the teardrop-shaped first wing 330 is truncated, making the first wing 330 more ovoid in shape. In this configuration, the anterior portion 138 of the first wing 330 can be longer than the truncated posterior end 336 of the first wing 330. As in previous embodiments, the spacer 120 can alternatively be a rotatable spacer rather than a fixed spacer. FIG. 9B illustrates a second wing 360 for use with such implants 300, the second wing 360 having a truncated posterior end 366. Truncation of the posterior ends 336,366 of the first and second wings 330,360 can reduce the possibility of interference of implants 300 having such first and second wings 330,360 positioned between spinous processes of adjacent pairs of cervical vertebrae, e.g., implants between cervical vertebrae five and six, and between cervical vertebrae six and seven. During rotation of the neck, the spinous process move past each other in a scissor-like motion. Each cervical vertebra can rotate relative to the next adjacent cervical vertebra in the general range of about 6°-12°. In addition, about 50 percent of the rotational movement of the neck is accomplished by the top two neck vertebrae. Thus, such embodiments can accommodate neck rotation without adjacent embodiments interfering with each other.

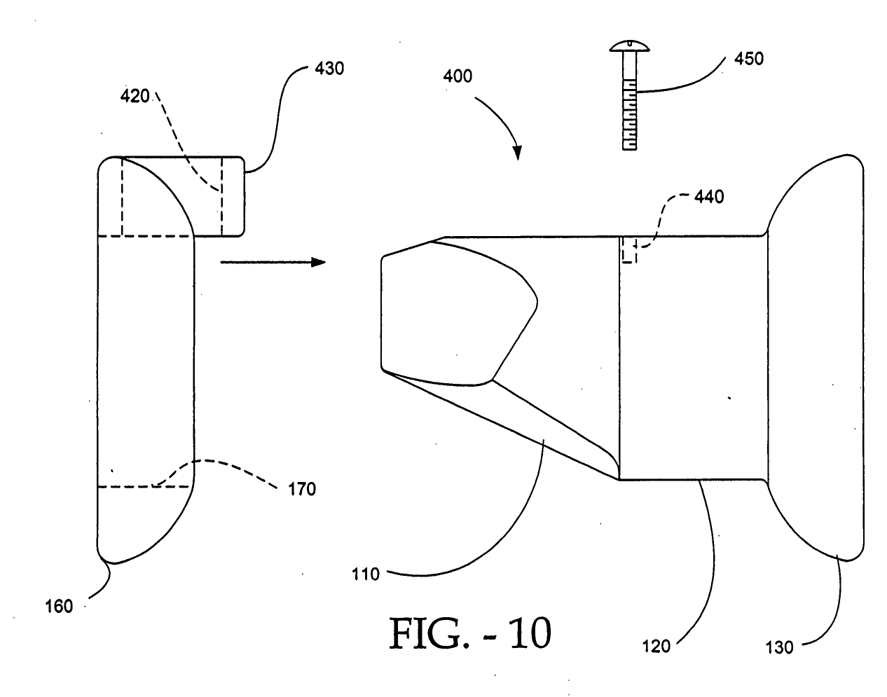

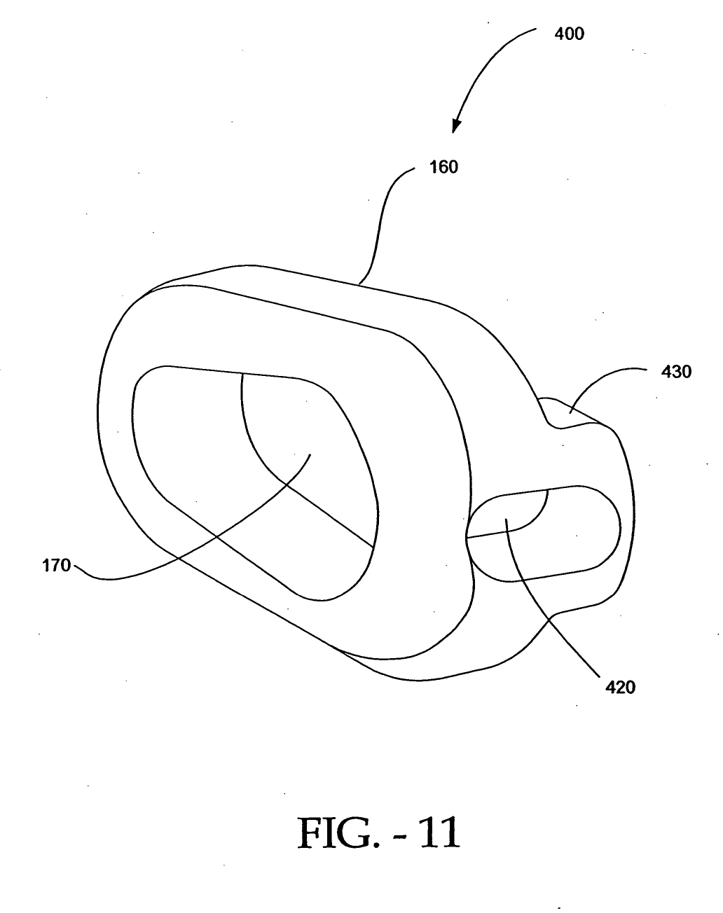

With respect to the prior embodiments which have first and second wings 130,160, the second wing 160, can be designed to be interference-fit onto the spacer 120 (where the spacer is fixed) or a portion of the distraction guide 110 adjacent to the spacer 120 (where the spacer is rotatable). Where the second wing 160 is interference-fit, there is no additional attachment device to fasten the second wing 160 relative to the remainder of the implant. Alternatively, various fasteners can be used to secure the second wing relative to the remainder of the implant. For example, FIGS. 10-12 illustrate an embodiment of an implant 400 including a teardrop-shaped second wing 460 having a bore 463 through a tongue 461 at the posterior end of the second wing 460. The bore 463 is brought into alignment with a corresponding bore 440 on the spacer 120 when the second wing 460 is brought into position by surgical insertion relative to the rest of the implant 400. A threaded screw 442 can be inserted through the aligned bores 463,440 in a posterior-anterior direction to secure the second wing 460 to the spacer 120. The direction of insertion from a posterior to an anterior direction has the screw 442 engaging the bores 463,440 and the rest of the implant 400 along a direction that is generally perpendicular to the longitudinal axis 125. This orientation is most convenient when the surgeon is required to use a screw 442 to secure the second wing 460 to the rest of the implant 400. Other securing mechanisms using a member inserted into corresponding bores 463,440 on the spacer 120 and second wing 460 are within the spirit of the invention. It should be understood that a rotatable spacer 220 also can be accommodated by this embodiment. With a rotatable spacer 220, the second wing 460 would be attached to a portion of the distraction guide 110 that is located adjacent to the rotatable spacer 220.

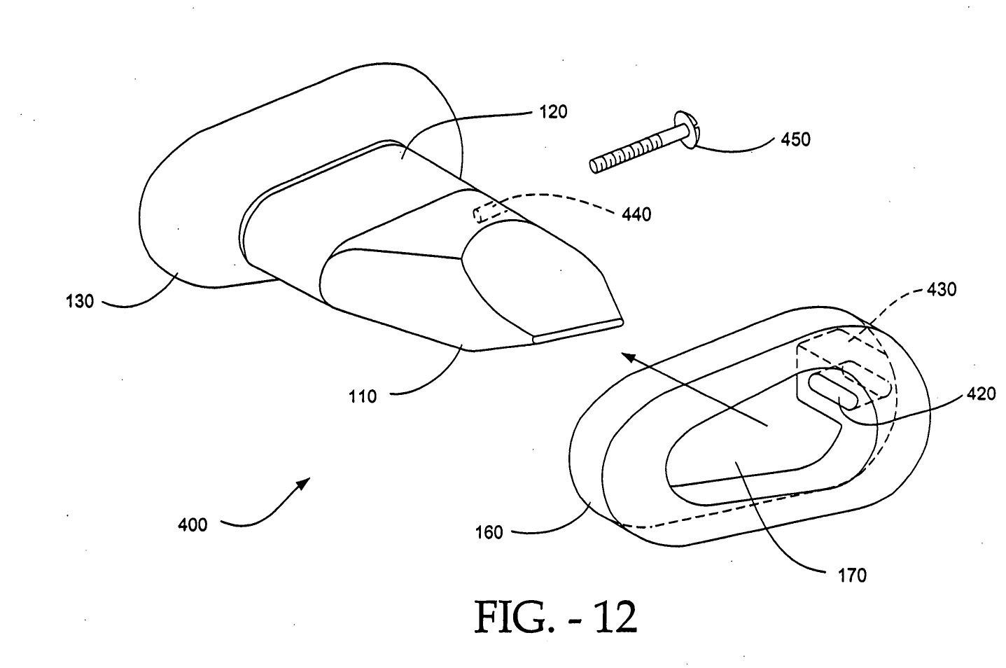

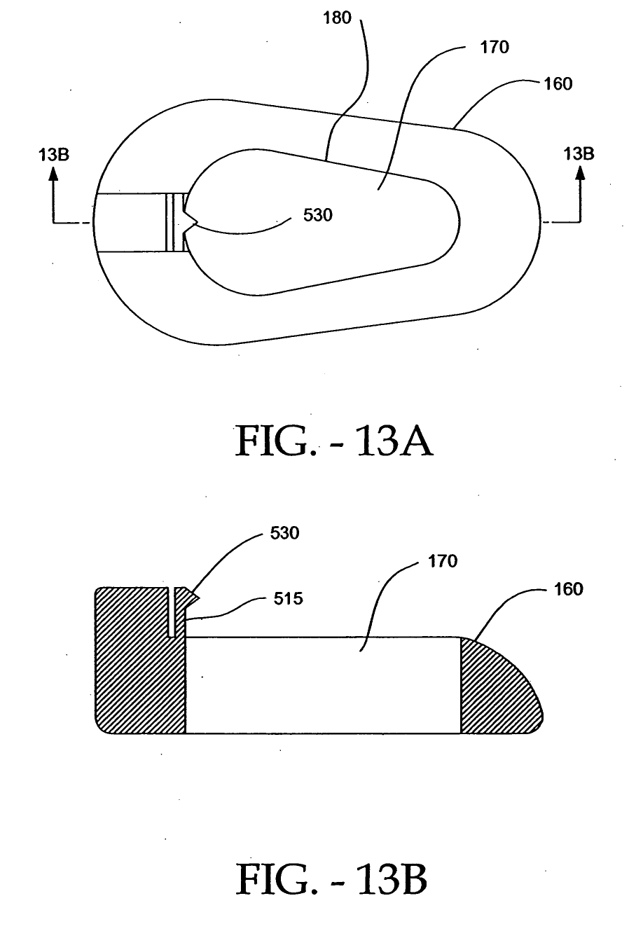

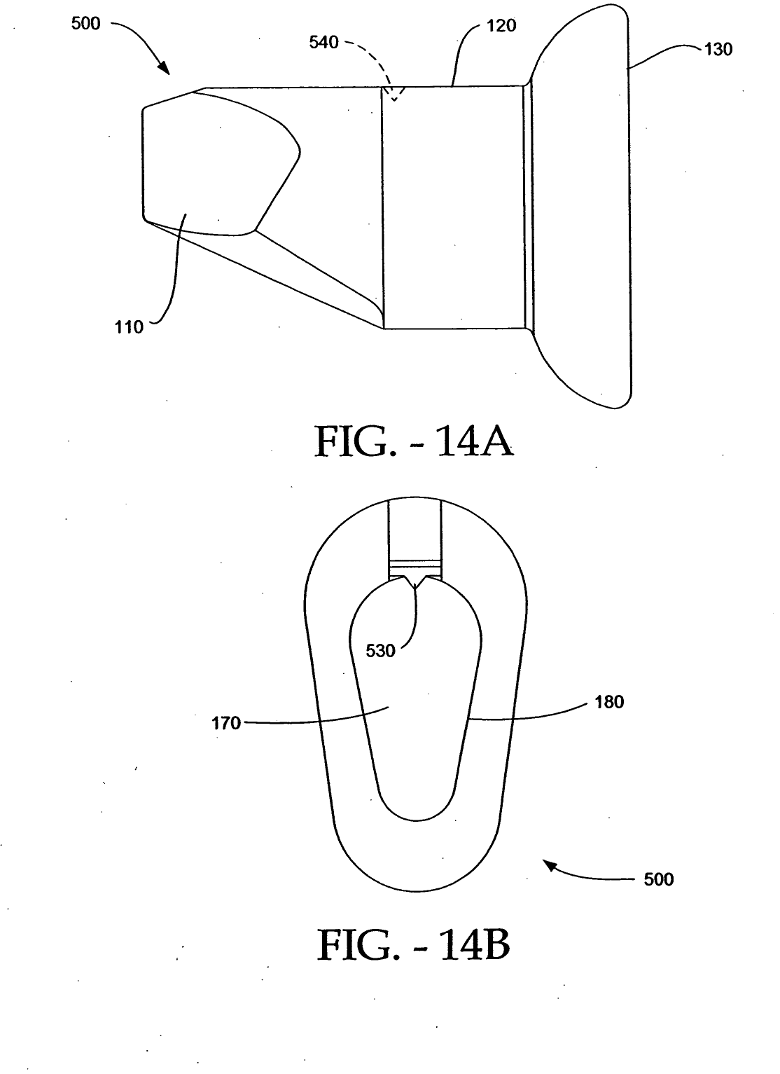

FIGS. 13A-14B depict a further embodiment 500 wherein the second wing 560 is secured to the spacer 120 by a mechanism including a flexible hinge 565, with a protrusion 561 on the end of the hinge 565 adjacent to the lip 562 of the opening 564 defined by portions of the second wing 560. The securing mechanism also encompasses an indentation 540 on the spacer 120, wherein the indentation 540 accommodates the protrusion 561 on the end of the flexible hinge 565. During surgery, after insertion of the distraction guide 110, spacer 120, and first wing 130, the second wing 560 is received over the distraction guide 110 and the spacer 120. As the second wing 560 is received by the spacer 120, the flexible hinge 565 and its protrusion 561 deflect until the protrusion 561 meets and joins with the indentation 540 in the spacer 120, securing the second wing 560 to the spacer 120. Again in embodiments where the spacer can rotate, the indentation 540 is located on an end of the distraction guide 110 that is adjacent to the rotatable spacer 220. With respect to the flexible hinge 565, this hinge is in a preferred embodiment formed with the second wing 560 and designed in such a way that it can flex as the hinge 565 is urged over the distraction guide 110 and the spacer 120 and then allow the protrusion 561 to be deposited into the indentation 540. Alternatively, it can be appreciated that the indentation 540 can exist in the second wing 560 and the flexible hinge 565 and the protrusion 561 can exist on the spacer 120 in order to mate the second wing 560 to the spacer 120. Still alternatively, the flexible hinge 565 can be replaced with a flexible protrusion that can be flexed into engagement with the indentation 540 in the embodiment with the indentation 540 in the spacer 120 or in the embodiment with the indentation 540 in the second wing 560. One of ordinary skill in the art will appreciate the myriad different ways with which the second wing can be mated with the implant.

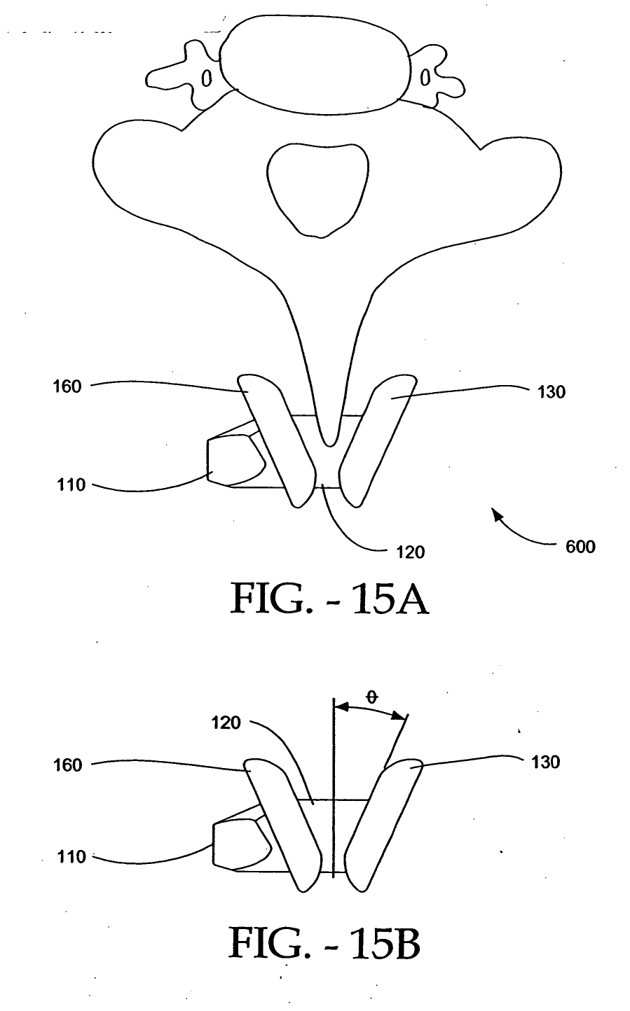

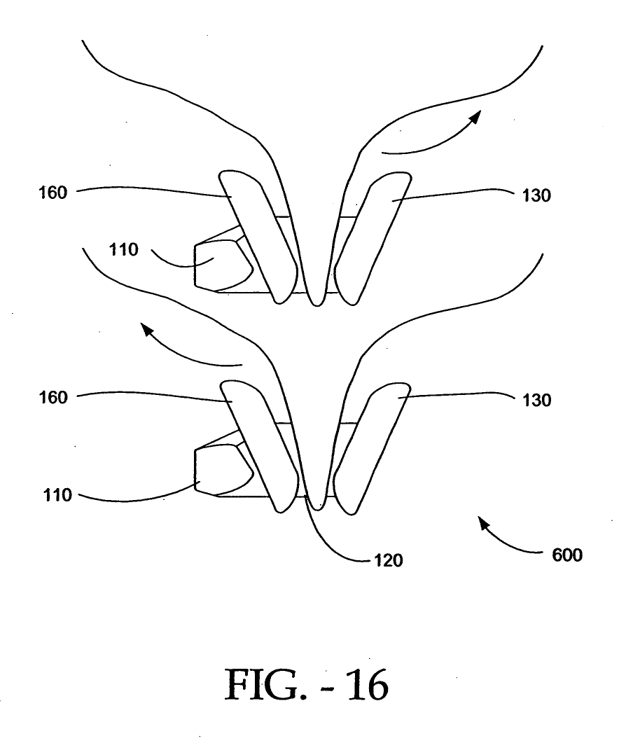

FIGS. 15A-16 illustrate an embodiment of an implant 600 wherein anterior ends of a first wing 630 and second wing 660 flare out at an angle away from the spacer 120 and away from each other. The cervical spinous processes are themselves wedge-shaped when seen from a top view. The first wing 630 and second wing 660 flare out so that the implant 600 can roughly conform with the wedge shape of the spinous processes, allowing the implant 600 to be positioned as close as possible to the vertebral bodies of the spine where the load of the spine is carried. The first and second wings 630,660 are positioned relative to the spacer, whether the spacer is fixed 120 or rotatable 220, so that the wings flare out as the wings approach the vertebral body of the spine. FIG. 15B is a top view of the implant 600 of FIG. 15A removed from proximity with the spinous processes. The first wing 630 is aligned at an angle with respect to an axis along the spinous processes perpendicular to the longitudinal axis (also referred to herein as the plane of symmetry). In one embodiment, the angle is about 30°, however, the angle θ can range from about 15° to about 45°. In other embodiments, other angles outside of this range are contemplated and in accordance with the invention. Likewise, the second wing 660 can be aligned along a similar, but oppositely varying range of angles relative to the plane of symmetry.

As described above in reference to FIG. 4, the second wing 660 defines an opening which is outlined by a lip. As is evident, the lip can be provided at an angle relative to the rest of the second wing 660 so that when the lip is urged into contact with the spacer 120, the second wing 660 has the desired angle relative to the spacer 120. As discussed above, there are various ways that the second wing 660 is secured to the spacer 120. FIG. 15A depicts a top view of one such implant 600 placed between the spinous processes of adjacent cervical vertebrae. FIG. 16 is a top view illustrating two layers of distracting implants 600 with flared wings 630,660.

Systems and methods in accordance with the present invention can include devices that can be used in cooperation with implants of the present invention. FIG. 17 illustrates “stops” (also referred to herein as “keeps”) 656, which are rings of flexible biocompatible material, which can be positioned around the spinous processes of adjacent cervical vertebrae and located posteriorly to the implant 600. The keeps 656 can prevent posterior displacement of implants. In one embodiment, the keeps can include a ring having a slit 658. The keeps 656 can be somewhat sprung apart, so that the keep 656 can be fit over the end of the spinous process and then allowed to spring back together in order to hold a position on the spinous process. The keep 656 can act as a block to the spacer 120 in order to prevent the implant 600 from movement in a posterior direction.

Interspinous Implant Having Slide-in Distraction Piece

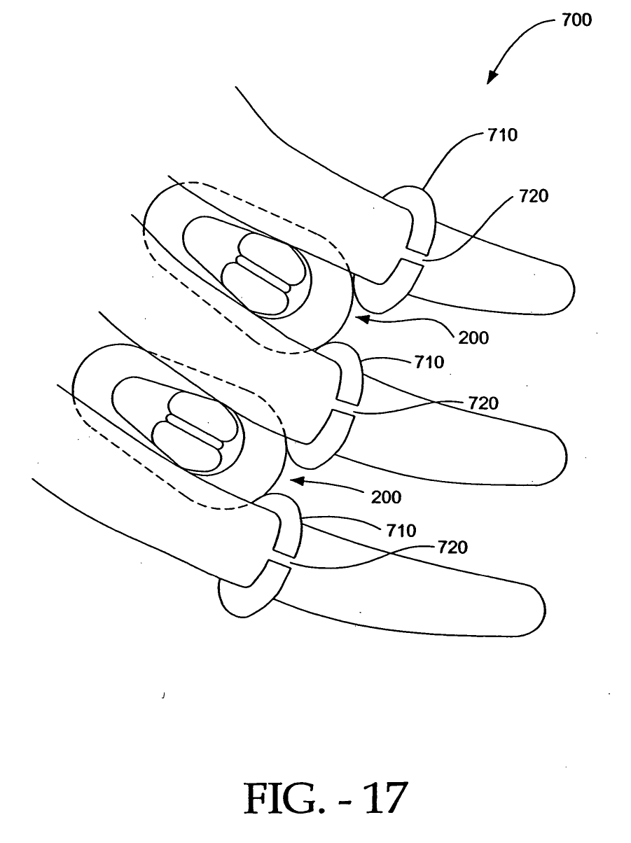

FIG. 18 is a perspective end view of an alternative embodiment of an implant 700 in accordance with the present invention. The implant 700 can include an initiating piece 704 and a slide-in distraction piece 702 adapted to be slidably coupled with the initiating piece 704. The initiating piece 704 and the slide-in distraction piece 702, when positioned between adjacent spinous processes and coupled together, can resemble implants 100 as described above with reference to FIGS. 1-17. For example, the implant 700 of FIG. 18 includes a first wing 730 at a distal end of the implant 700, a fixed spacer 720 extending from the first wing 730, a second wing 760 extending from the spacer 720 so that the spacer 720 is disposed between the first wing 730 and the second wing 760, and a distraction guide 710 at a proximal end 716 of the implant 700.

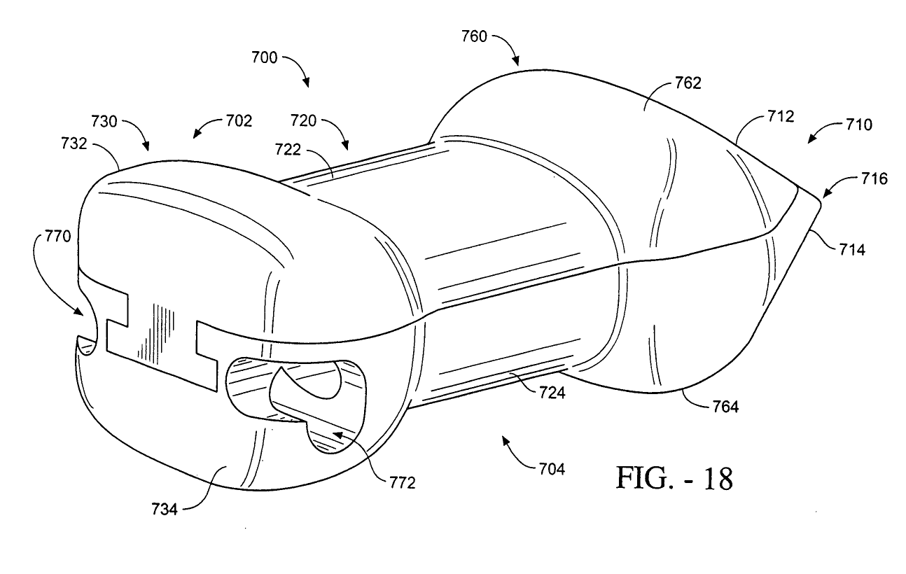

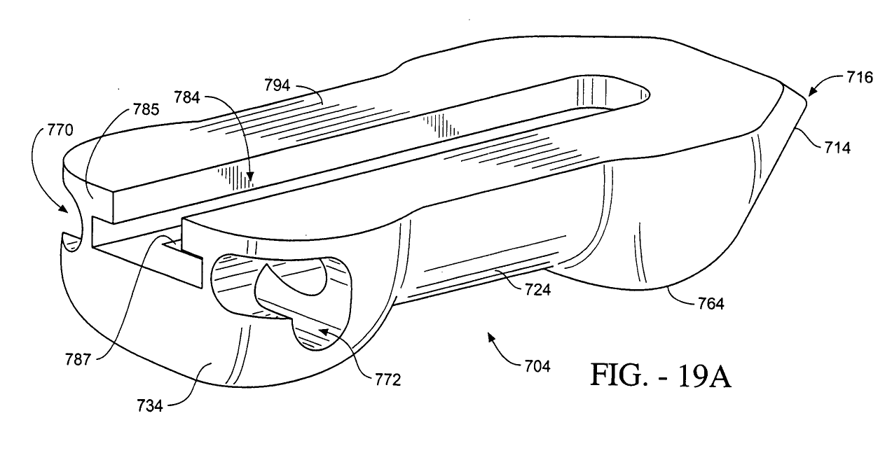

FIG. 19A is a perspective view of the initiating piece 704. The initiating piece 704 includes a slot 784 within a lower sliding surface 794 that extends through a substantial portion of the length of the initiating piece 704, the slot 784 being adapted to receive a rail 782 of the slide-in distraction piece 702. The slot 784 extends a length at least as long as the rail 782 and preferably does not extend through the entire initiating piece 704 so that the distraction piece 702 is prevented from sliding out of position in the direction of insertion. As shown, the slot 784 includes a flange 785 along the periphery of the slot 784 to retain the rail 782 within the slot 784. The slot 784 is thus shaped to substantially conform with a “T” shaped cross-section of the rail 782 so that when the slide-in distraction piece 702 is mated with the initiating piece 704 and the rail 782 is seated within the slot 784, relative movement between the distraction piece 702 and the initiating piece 704 is limited or substantially blocked, except along the longitudinal axis 725 in a direction opposite the direction of insertion. To limit or block movement along the longitudinal axis 725 in a direction opposite the direction of insertion, the slot 784 can include a recess 787 adapted to receive a catch 781 of the rail 782 so that when the catch 781 passes over the recess 787, the catch 781 is extended, locking the distraction piece 702 in place, and limiting or blocking movement in a direction opposite insertion. Alternatively, the catch 781 can be extendably associated with the slot 784, while the recess 787 is formed within the rail 782 for receiving the catch 781.

The initiating piece 704 includes a lower distraction element 714 having a contact surface that tapers to the proximal end 716 from above as well as below the proximal end 716 so that the lower distraction element 714 has a “V” shape in cross-section along an axis of the spine. Such a geometry can ease implantation when compared with a distraction element 714 that tapers to the proximal end only from below (or above) the proximal end 716 by more evenly distributing a load force applied to the lower distraction element 714 by the interspinous ligament 6 during initial piercing and/or distraction of the interspinous ligament 6. The initiating piece 704 further includes a lower portion 734 of the first wing, a lower portion 764 of the second wing, and a lower portion 724 of the spacer. In an embodiment, the lower portions 734,764,724 can be integrally formed as the lower distraction element 714, thereby avoiding discontinuities in a lower sliding surface 794 of the initiation piece 704. The lower sliding surface 794 of the initiating piece 704 is substantially flat and preferably smooth to ease receipt of the rail 782 within the slot 784. The lower sliding surface 794 slopes upward relative to the longitudinal axis 725 from the distal end of the initiating piece 704 to the proximal end of the initiating piece 704. The slope of the lower sliding surface 794 causes variation in thickness of the lower portion 724 of the spacer from the distal end of the spacer to the proximal end of the spacer. This slope aids in the distraction of the spinous processes upon insertion of the distraction piece 702.

Referring again to FIG. 18, the contact surfaces of the implant 700 include relatively smooth transitions from the distraction guide 710 to the second wing 760, and from the second wing 760 to the spacer 720. As described in greater detail below, during implantation the initiating piece 704 and the distraction piece 702 are positioned as separate, single pieces. A relatively continuous surface with smooth transitions improves ease of implantation and minifies obstruction of the initiating piece 704 and the distraction piece 702 by the adjacent spinous processes and/or related tissues. In contrast to implants as described with reference to FIGS. 1-17, it is preferable that the distraction piece 702 and the initiating piece 704 have smoother transitions between the distraction guide 710, the second wing 760, and the spacer 720, as such transitions even further lessen the obstruction to the movement of the implant during implantation.

The lower portion 734 of the first wing can further optionally include one or more cavities 770 for receiving prongs of an insertion tool. As shown in FIGS. 19A through 19C, the initiating piece 704 includes two cavities 770 extending from the distal end of the initiating piece 704 toward the proximal end 716, with one cavity 770 being arranged on each side of the lower portion 734 of the first wing. Each cavity 770 can be sized to receive a prong of the insertion tool. The cavity 770 can further include a groove 772 extending perpendicular to the cavity 770. Referring to FIGS. 19B and 19C, a prong 795 of an insertion tool 794 can include, in an embodiment, a protrusion 796 that fits within the groove 772. When the prong is inserted into the cavity 770 and rotated approximately 90 degrees (FIG. 19C) so that the protrusion is rotated into the groove 772, the prong is “locked” within the cavity 770. Once the prongs of the insertion tool are arranged in a locked configuration, the implant 700 can be releasably guided into position between the adjacent spinous processes.

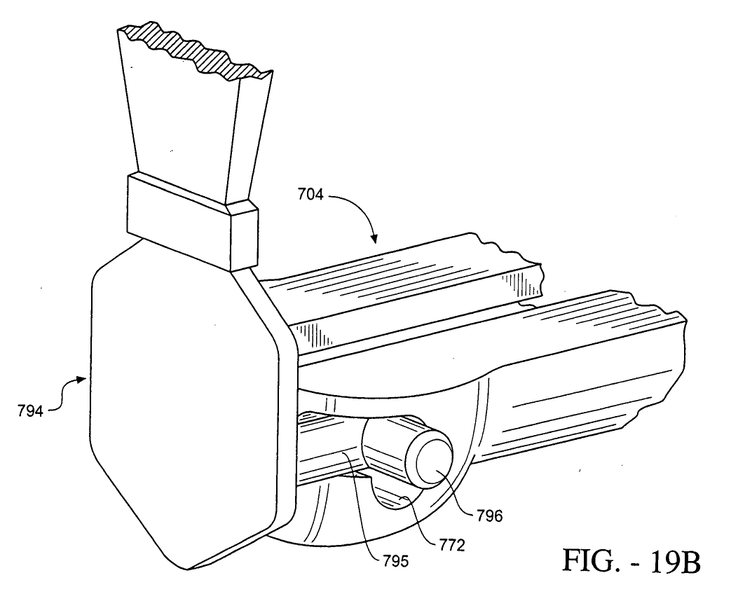

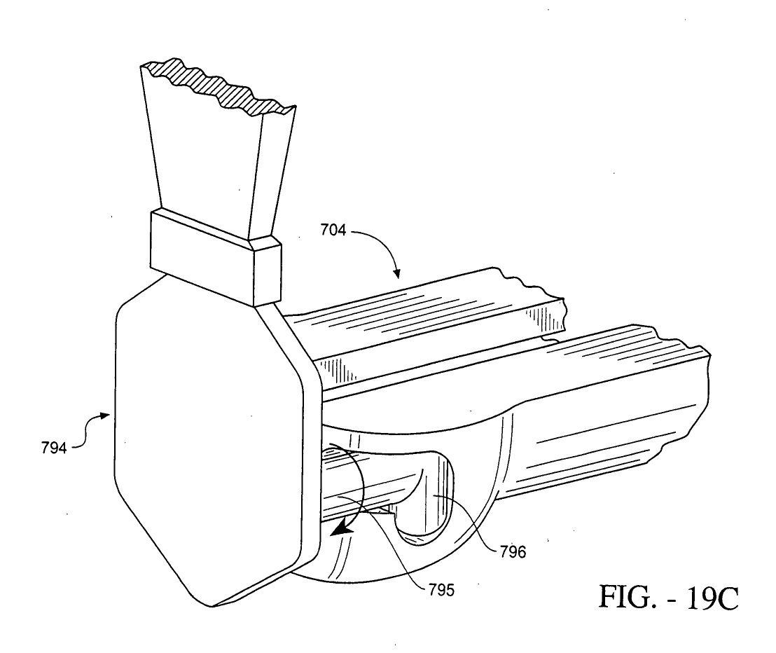

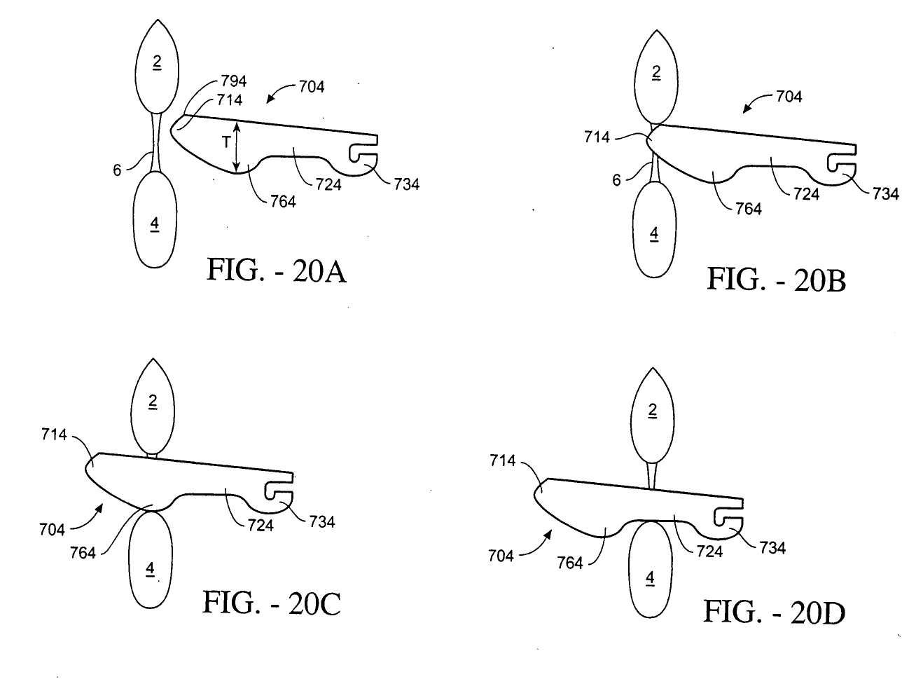

FIG. 20A is a posterior view of the initiating piece 704 positioned adjacent to the interspinous ligament 6. As can be seen, the initiating piece 704 has a maximum thickness T from the lower sliding surface 794 to the lower portion 764 of the second wing. In a preferred embodiment, the maximum thickness T of the initiating piece 704 is approximately the same as, or less than the thickness of the spacer 720 when the initiating piece 704 and the distraction piece 702 are mated and the implant 700 is positioned between the adjacent spinous processes 2,4. Referring to FIG. 20B, as the initiating piece 704 is urged into the interspinous ligament 6, the lower distraction element 714 pierces and/or distracts the fibers of the interspinous ligament 6. As shown in FIG. 2C, the initiating piece 704 is further urged through the interspinous ligament 6 so that the lower portion 764 of the second wing passes between the adjacent spinous processes 2,4 but preferably does not distract the space between the adjacent spinous processes 2,4 beyond the maximum distraction height of the spacer 720. As shown in FIG. 20D, the initiating piece 704 is further urged through the interspinous ligament 6 so that the lower portion 724 of the spacer is approximately positioned between the adjacent spinous processes 2,4. Note that in other embodiments, the maximum thickness T from the lower sliding surface 794 to the lower portion 764 of the second wing can be greater than the ultimate thickness of the spacer 720 so that when the initiating piece 704 is positioned between adjacent spinous processes 2,4, the space between the spinous processes 2,4 is distracted to a height greater than the distraction height of the spacer 720. In such embodiments, the second wing 760 can potentially provide greater range of flexion motion (wherein the space between adjacent spinous processes increases) while assuring that the movement of the implant 700 will be limited or blocked in a direction opposite insertion by the second wing 760.

FIG. 21 is a flipped perspective end view of the slide-in distraction piece 702. The distraction piece 702 includes a rail 782 extending over a substantial portion of the length of the distraction piece 702, roughly corresponding to a length of the slot 784 of the initiating piece 704, within which the rail 782 is adapted to be received. The height of the rail 782 from the upper sliding surface 792 to the flange 783 of the rail 782 approximately corresponds to the depth of the slot 784 from the lower sliding surface 794 to the bottom of the flange 785 of the slot, so that when the rail 782 is received within the slot 784, the upper sliding surface 792 of the distraction piece 702 is substantially flush with the lower sliding surface 794. In other embodiments, a gap can exist between the upper sliding surface 792 and the lower sliding surface 794. As described above, the surface of the rail 782 includes a catch 781 arranged along the length of the rail 782 so that the catch 781 roughly corresponds to the recess 787 disposed within the slot 784. The catch 781 can have a sloped leading edge (from the proximal end to a distal end of the catch 781) and can be spring loaded, or otherwise biased so that the catch 781 collapses when the distraction piece 702 slides along the lower sliding surface 794 of the initiating piece 704 and extends when passing over the recess 787. The catch 781 can have a trailing edge substantially perpendicular to the slot 784 so that the catch 781 resists movement of the distraction piece 702 in a direction opposite insertion. In other embodiments, the catch 781 can be some other mechanism. For example, in an alternative embodiment, the catch 781 can be a flexible hinge and protrusion similar in operation to that described in FIGS. 13A-14B. Still further the pieces 702,704 can be flexible enough that the catch 781 is molded into the piece 702,704 and can snap into the recess 787 in the other piece 702,704.

The distraction piece 702 includes an upper distraction element 712 having a contact surface that tapers so that the upper distraction element 712 has a ramp shape. The distraction piece 702 further includes an upper portion 732 of the first wing, an upper portion 762 of the second wing, and an upper portion 722 of the spacer. In an embodiment, the upper portions 732,762,722 can be integrally formed with the upper distraction element 712, thereby avoiding discontinuities in an upper sliding surface 792 of the distraction piece 702. As with the lower sliding surface 790, the upper sliding surface 792 of the distraction piece 702 is substantially flat and preferably smooth to ease positioning of the rail 782 within the slot 784. The upper sliding surface 792 slopes upward relative to the longitudinal axis 725 from the distal end of the distracting piece 702 to the proximal end of the distraction piece 702, the slope of the upper sliding surface 792 being substantially similar to the slope of the lower sliding surface 794 so that the two surfaces 792,794 are substantially parallel, and mate when the rail 782 is positioned within the slot 784. The slope of the upper sliding surface 792 causes variation in thickness of the upper portion 722 of the spacer from the distal end of the spacer to the proximal end of the spacer so that the upper portion 722 of the spacer is thicker at the distal end. When the distraction piece 702 is mated with the initiating piece 704 so that the rail 782 is seated within the slot 784, the thickness of the spacer 720 is approximately the same across the length of the spacer 720.

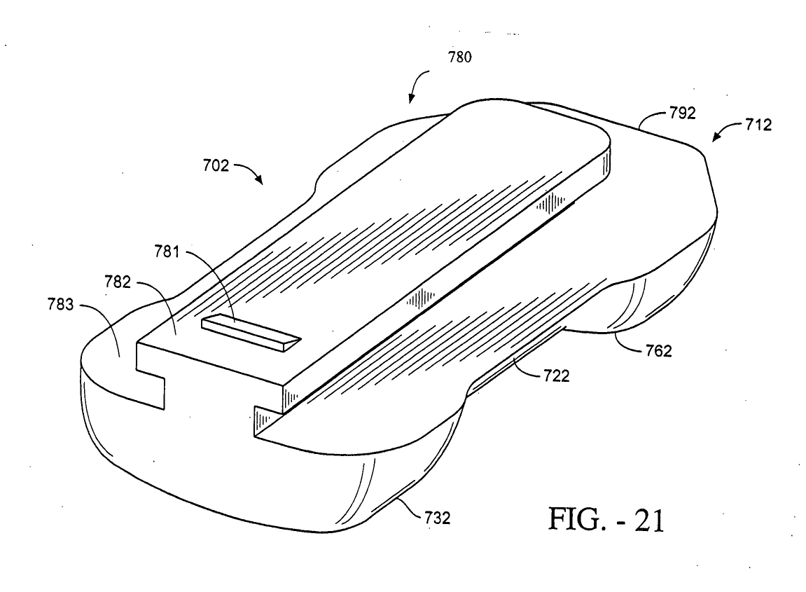

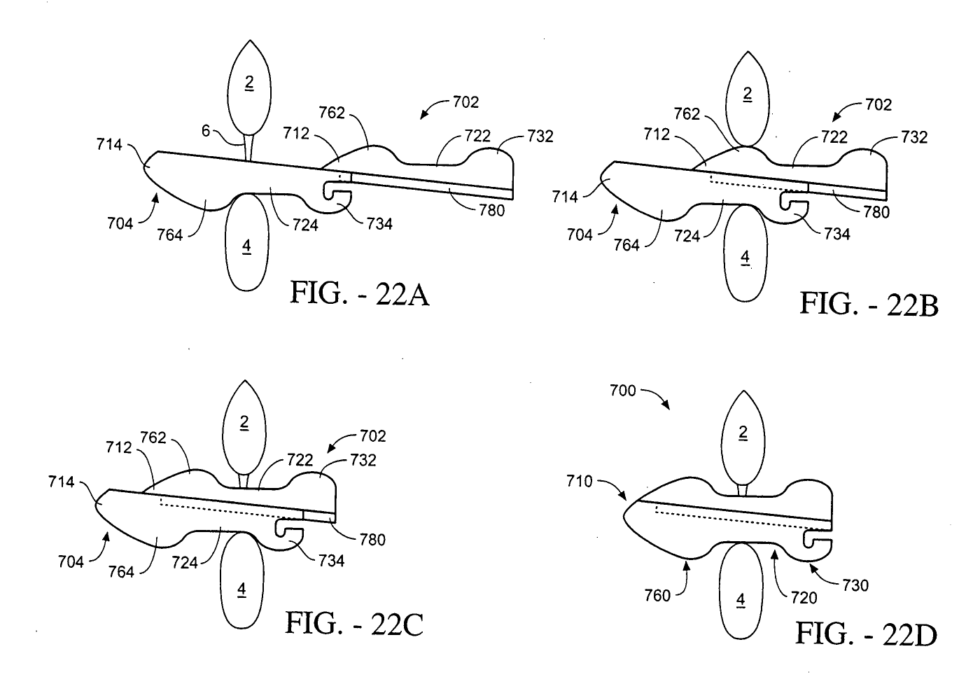

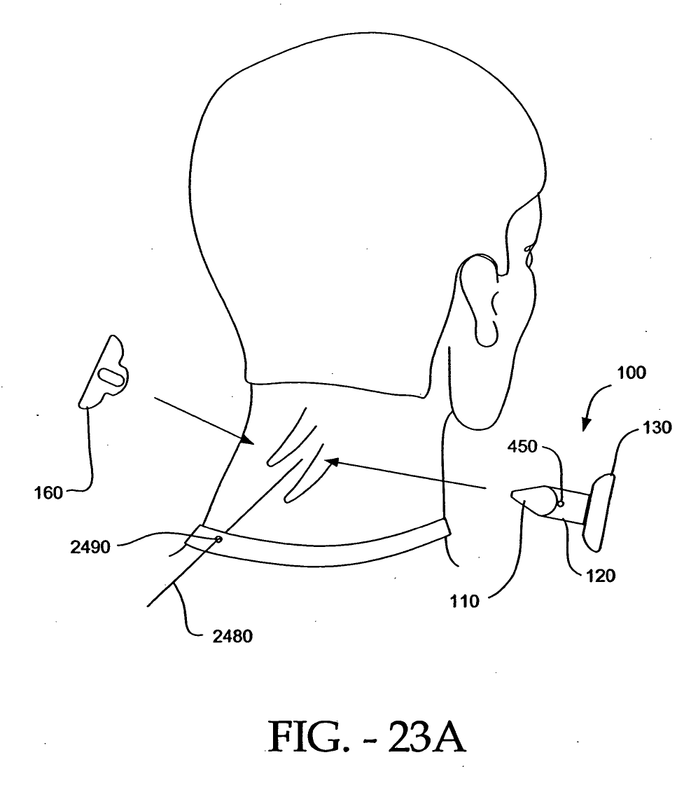

FIGS. 22A through 22D are a series of posterior views of the distraction piece 702 mating with the initiating piece 704 so that the implant 700 is positioned between adjacent spinous processes 2,4 to support a load applied by the adjacent spinous processes 2,4 during an extension motion. As can be seen, the distraction piece 702 is positioned so that the proximal end of the rail flange 783 fits within the slot 784. The distraction piece 702 can then be urged toward the interspinous ligament 6 so that the rail 782 is further received within the slot 784. The thickness of the implant 700 increases as the initiating piece 704 is mated with the distraction piece 702. FIG. 22B illustrates the distraction piece 702 arranged so that the upper distraction element 782 is adjacent to the interspinous ligament 6. As the distraction piece 702 is urged further toward the interspinous ligament 6, the upper distraction element 782 wedges between the lower sliding surface 794 and the interspinous ligament 6 and/or the adjacent spinous processes 2,4, gradually distracting the interspinous ligament 6 and the adjacent spinous processes 2,4 as the distraction piece 702 is further urged in the direction of insertion. As shown in FIG. 22C, as the upper portion 762 of the second wing passes between the adjacent spinous processes 2,4, the space between the adjacent spinous processes 2,4 is distracted beyond the maximum distraction height of the spacer 720. The distraction piece 702 is further urged in the direction of insertion until the rail 782 is seated within the slot 784 and the upper portion 762 of the second wing is arranged so that the interspinous ligament 6 and/or adjacent spinous processes 2,4 are disposed between the upper portion 762 of the second wing and the upper portion 732 of the first wing (see FIG. 22D). As the catch 781 passes over the recess 787, the catch 781 extends into the recess 787, locking the distraction piece 702 in position, mated with the initiation piece 704.

Materials for Use in Implants of the Present Invention

In some embodiments, the implant can be fabricated from medical grade metals such as titanium, stainless steel, cobalt chrome, and alloys thereof, or other suitable implant material having similar high strength and biocompatible properties. Additionally, the implant can be at least partially fabricated from a shape memory metal, for example Nitinol, which is a combination of titanium and nickel. Such materials are typically radiopaque, and appear during x-ray imaging, and other types of imaging. Implants in accordance with the present invention, and/or portions thereof can also be fabricated from somewhat flexible and/or deflectable material. In these embodiments, the implant and/or portions thereof can be fabricated in whole or in part from medical grade biocompatible polymers, copolymers, blends, and composites of polymers. A copolymer is a polymer derived from more than one species of monomer. A polymer composite is a heterogeneous combination of two or more materials, wherein the constituents are not miscible, and therefore exhibit an interface between one another. A polymer blend is a macroscopically homogeneous mixture of two or more different species of polymer. Many polymers, copolymers, blends, and composites of polymers are radiolucent and do not appear during x-ray or other types of imaging. Implants comprising such materials can provide a physician with a less obstructed view of the spine under imaging, than with an implant comprising radiopaque materials entirely. However, the implant need not comprise any radiolucent materials.

One group of biocompatible polymers are the polyaryletherketone group which has several members including polyetheretherketone (PEEK), and polyetherketoneketone (PEKK). PEEK is proven as a durable material for implants, and meets the criterion of biocompatibility. Medical grade PEEK is available from Victrex Corporation of Lancashire, Great Britain under the product name PEEK-OPTIMA. Medical grade PEKK is available from Oxford Performance Materials under the name OXPEKK, and also from CoorsTek under the name BioPEKK. These medical grade materials are also available as reinforced polymer resins, such reinforced resins displaying even greater material strength. In an embodiment, the implant can be fabricated from PEEK 450G, which is an unfilled PEEK approved for medical implantation available from Victrex. Other sources of this material include Gharda located in Panoli, India. PEEK 450G has the following approximate properties:

| Property | Value | |
|---|---|---|
| Density | 1.3 | g/cc |
| Rockwell M | 99 | |
| Rockwell R | 126 | |
| Tensile Strength | 97 | MPa |
| Modulus of Elasticity | 3.5 | GPa |
| Flexural Modulus | 4.1 | GPa |

PEEK 450G has appropriate physical and mechanical properties and is suitable for carrying and spreading a physical load between the adjacent spinous processes. The implant and/or portions thereof can be formed by extrusion, injection, compression molding and/or machining techniques.

It should be noted that the material selected can also be filled. Fillers can be added to a polymer, copolymer, polymer blend, or polymer composite to reinforce a polymeric material. Fillers are added to modify properties such as mechanical, optical, and thermal properties. For example, carbon fibers can be added to reinforce polymers mechanically to enhance strength for certain uses, such as for load bearing devices. In some embodiments, other grades of PEEK are available and contemplated for use in implants in accordance with the present invention, such as 30% glass-filled or 30% carbon-filled grades, provided such materials are cleared for use in implantable devices by the FDA, or other regulatory body. Glass-filled PEEK reduces the expansion rate and increases the flexural modulus of PEEK relative to unfilled PEEK. The resulting product is known to be ideal for improved strength, stiffness, or stability. Carbon-filled PEEK is known to have enhanced compressive strength and stiffness, and a lower expansion rate relative to unfilled PEEK. Carbon-filled PEEK also offers wear resistance and load carrying capability.

As will be appreciated, other suitable similarly biocompatible thermoplastic or thermoplastic polycondensate materials that resist fatigue, have good memory, are flexible, and/or deflectable, have very low moisture absorption, and good wear and/or abrasion resistance, can be used without departing from the scope of the invention. As mentioned, the implant can be comprised of polyetherketoneketone (PEKK). Other material that can be used include polyetherketone (PEK), polyetherketoneetherketoneketone (PEKEKK), polyetheretherketoneketone (PEEKK), and generally a polyaryletheretherketone. Further, other polyketones can be used as well as other thermoplastics. Reference to appropriate polymers that can be used in the implant can be made to the following documents, all of which are incorporated herein by reference. These documents include: PCT Publication WO 02/02158 A1, dated Jan. 10, 2002, entitled “Bio-Compatible Polymeric Materials;” PCT Publication WO 02/00275 A1, dated Jan. 3, 2002, entitled “Bio-Compatible Polymeric Materials;” and, PCT Publication WO 02/00270 A1, dated Jan. 3, 2002, entitled “Bio-Compatible Polymeric Materials.” Other materials such as Bionate®, polycarbonate urethane, available from the Polymer Technology Group, Berkeley, Calif., may also be appropriate because of the good oxidative stability, biocompatibility, mechanical strength and abrasion resistance. Other thermoplastic materials and other high molecular weight polymers can be used.

It is to be understood that embodiments in accordance with the present invention can be constructed without a pliant material. It is also to be understood that the embodiments in accordance with the present invention can have other dimensions.

Methods for Implanting Interspinous Implants

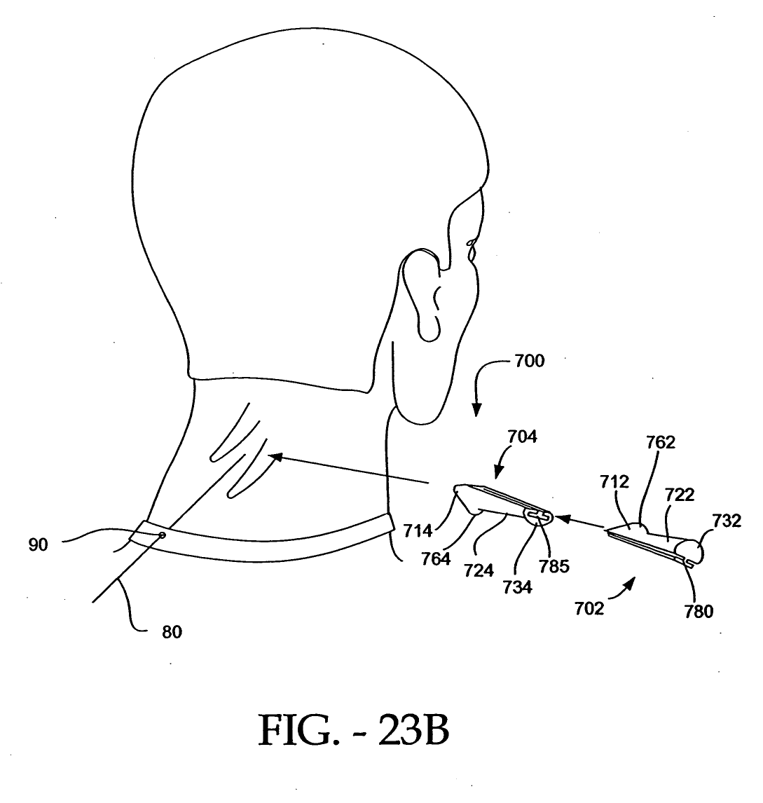

A minimally invasive surgical method for implanting an implant 400 in the cervical spine is disclosed and taught herein. In this method, as shown in FIG. 23A, preferably a guide wire 80 is inserted through a placement network or guide 90 into the neck of the implant recipient. The guide wire 80 is used to locate where the implant is to be placed relative to the cervical spine, including the spinous processes. Once the guide wire 80 is positioned with the aid of imaging techniques, an incision is made on the side of the neck so that an implant in accordance with an embodiment of the present invention, can be positioned in the neck thorough an incision and along a line that is about perpendicular to the guide wire 80 and directed at the end of the guide wire 80. In one embodiment, the implant can be a sized implant 400 (i.e., having a body that is not distractable), such as described above in FIGS. 1-17 and including a distraction guide 110, a spacer 120, and a first wing 130. The implant 400 is inserted into the neck of the patient. Preferably during insertion, the distraction guide 110 pierces or separates the tissue without severing the tissue.

Once the implant 400 is satisfactorily positioned, a second wing 460 can be optionally inserted along a line that is generally colinear with the line over which the implant 400 is inserted but from the opposite side of the neck. The anatomy of the neck is such that it is most convenient and minimally invasive to enter the neck from the side with respect to the implant 400 and the second wing 460. The second wing 460 is mated to the implant and in this particular embodiment, the second wing 460 is attached to the implant 400 by the use of a fastener, for example by a screw 442. Where a screw is used, the screw 442 can be positioned using a screw driving mechanism that is directed along a posterior to anterior line somewhat parallel to the guide wire 80. This posterior to anterior line aids the physician in viewing and securing the second wing 460 to the implant. The second wing 460 is positioned so that a bore 463 formed in a lip 461 of the second wing 460 is aligned with a bore 440 of the implant 400, as described above. The screw 442 is positioned within both bores and secured, at least, to the bore 440 of the implant 400. In other embodiments, the second wing can be interference fit with the implant, as described above, or fastened using some other mechanism, such as a flexible hinge and protrusion.

In other embodiments of methods in accordance with the present invention, the implant can include an initiating piece 704 and a distraction piece 702, such as described above in FIGS. 18-22D. In such embodiments, as shown in FIG. 23B, preferably a guide wire 80 is inserted through a placement network or guide 90 into the neck of the implant recipient (as shown and described above). Once the guide wire 80 is positioned with the aid of imaging techniques, an incision is made on the side of the neck so that an initiating piece 704 of the implant 700 can be positioned in the neck thorough an incision and along a line that is about perpendicular to the guide wire 80 and directed at the end of the guide wire. The initiating piece 704 can include a lower distraction element 714, a lower portion 764 of the second wing, a lower portion 724 of the spacer, and a lower portion 734 of the first wing. The implant 700 is inserted into the neck of the patient, between adjacent spinous processes. Preferably during insertion, the lower distraction element 714 pierces or separates the tissue without severing the tissue, and the implant 700 is positioned so that the upper portion 724 of the spacer is disposed between the adjacent spinous processes.

Once the initiating piece 704 is satisfactorily positioned, a distracting piece 702 can be inserted along a line that is approximately colinear with the line over which the initiating piece 704 is inserted, but positioned so that a rail 782 of the distracting piece 702 mates with a slot 784 of the initiating piece 704. The anatomy of the neck is such that it is most convenient and minimally invasive to enter the neck from the side with respect to the implant 700. The distracting piece 702 can be mated to the initiating piece 704 through an interference fit, or using a catch 781 and recess 787 as described above, alternatively by connecting the distracting piece 704 with the initiating piece 702 using a fastener, or by some other device, as described above. It is to be understood that the embodiment described herein can be used between any of the spinous processes of the spine.

The foregoing description of the present invention have been presented for purposes of illustration and description. It is not intended to be exhaustive or to limit the invention to the precise forms disclosed. Many modifications and variations will be apparent to practitioners skilled in this art. The embodiments were chosen and described in order to best explain the principles of the invention and its practical application, thereby enabling others skilled in the art to understand the invention for various embodiments and with various modifications as are suited to the particular use contemplated. It is intended that the scope of the invention be defined by the following claims and their equivalents.

What is claimed:

1. An interspinous implant adapted to be arranged between spinous processes, the implant comprising:
an initiating piece; and
a distraction piece that can be slidably associated with the initiating piece so that the distraction piece is disposed adjacent the initiating piece;
wherein the initiating piece is adapted to be arranged between the spinous processes before the distraction piece is disposed over the initiating piece.

2. The implant of claim 1, further comprising:
a cavity disposed within at least a portion of the initiating piece;
a protrusion extending from at least a portion of the distraction piece; and
wherein when the distraction piece is slidably associated with the initiating piece, the protrusion is received within the cavity.

3. The implant of claim 2, wherein:
the cavity is a slot having a flange extending from a periphery of the slot; and
the protrusion is a rail having a flange extending from a periphery of the rail.

4. The implant of claim 1, wherein:
the initiating piece includes a sliding surface against which the distraction piece can slide and a contact surface adapted to contact one of the spinous processes; and
the sliding surface is arranged at a non-zero angle relative to the contact surface.

5. The implant of claim 4, wherein:
the sliding surface of the initiating piece is a first sliding surface;
the distraction piece includes a second sliding surface against which the first sliding surface is adapted to slide; and
the second sliding surface is adapted to be arranged substantially parallel to the first sliding surface when disposed between the spinous processes.

6. The implant of claim 5 wherein:
the contact surface of the initiating piece is a first contact surface; and
the distraction piece includes a second contact surface adapted to contact the other of the spinous processes; and
when the distraction piece is disposed over the initiating piece, a spacer is defined between the first contact surface and the second contact surface.

7. The implant of claim 1, wherein:
the initiating piece includes a lower distraction element, a lower portion of a first wing, a lower portion of a spacer, and a lower portion of a second wing; and
the distraction piece includes an upper distraction element, an upper portion of the first wing, an upper portion of the spacer, and an upper portion of the second wing; and
when the distraction piece is disposed over the initiating piece, the spacer is disposed between the first wing and the second wing.

8. The implant of claim 7, wherein one or both of the first wing and the second wing are adapted to limit movement of the implant relative to the spinous processes.

9. The implant of claim 3, wherein the rail includes a catch and the slot includes a recess so that when the catch is received within the recess, relative movement of the initiating piece and the distraction piece is limited.

10. An interspinous implant adapted to be arranged between spinous processes, the interspinous implant having a first wing at a distal end of the interspinous implant, a second wing, a spacer disposed between the first wing and the second wing, and a distraction guide at the proximal end of the interspinous implant, wherein the improvement comprises:
the implant includes an initiating piece and a distraction piece adapted to be slidably associated with one another;
wherein the initiating piece includes an initiating sliding surface and an initiating contact surface, the initiating contact surface having a first portion of the first wing, a first portion of the spacer, a first portion of the second wing and a first portion of the distraction guide; and
wherein the distraction piece includes a distraction sliding surface and a distraction contact surface, the distraction contact surface having a second portion of the first wing, a second portion of the spacer, a second portion of the second wing and a second portion of the distraction guide.

11. The implant of claim 10, wherein:
the initiating sliding surface is arranged at a non-zero angle relative to the first portion of the spacer; and
the distraction sliding surface is adapted to be arranged substantially parallel to the initiating sliding surface when the implant is disposed between the spinous processes.

12. The implant of claim 10, further comprising:
a cavity disposed within at least a portion of the initiating sliding surface;
a protrusion extending from at least a portion of the distraction sliding surface; and
wherein when the distraction piece is slidably associated with the initiating piece, the protrusion is received within the cavity.

13. The implant of claim 12, wherein:
the cavity is a slot having a flange extending from a periphery of the slot; and
the protrusion is a rail having a flange extending from a periphery of the rail.

14. The implant of claim 13, wherein the rail includes a catch and the slot includes a recess so that when the catch is received within the recess, relative movement of the initiating piece and the distraction piece is limited.

15. The implant of claim 1, wherein:
the initiating piece including a first sliding surface and a first contact surface; and
the distraction piece including a second sliding surface and a second contact surface, the second sliding surface being adapted to slide along the first sliding surface so that the distraction piece is disposed over the initiating piece.

16. The implant of claim 15, further comprising:
a cavity disposed within at least a portion of the first sliding surface;
a protrusion extending from at least a portion of the second sliding surface; and
wherein when the distraction piece slides along the initiating piece, the protrusion is received within the cavity.

17. The implant of claim 16, wherein:
the cavity is a slot having a flange extending from a periphery of the slot; and
the protrusion is a rail having a flange extending from a periphery of the rail.

18. The implant of claim 15, wherein:
the first sliding surface is arranged at a non-zero angle relative to the first contact surface; and
the second sliding surface is adapted to be arranged substantially parallel to the first sliding surface when disposed between the spinous processes.

19. A method of arranging an interspinous implant between spinous processes, the implant having a first wing, a second wing, and a spacer disposed between the first wing and the second wing, the method comprising:
using the implant, the implant including an initiating piece and a distraction piece adapted to be slidably associated with the initiating piece, the initiating piece and the distraction piece each having a portion of a first wing, a portion of a second wing, and a portion of a spacer disposed between the first wing and the second wing;
urging the initiating piece between the spinous processes so that the portion of the spacer is disposed between the spinous processes;
slidably associating the distraction piece with the initiating piece so that the distraction piece is disposed over the initiating piece such that the spacer is disposed between the spinous processes.

20. The method of claim 20,
wherein:
the initiating piece includes a cavity disposed therein; and
the distraction piece includes a protrusion extending therefrom; and
further comprising:
arranging the protrusion within the cavity.
